(19) United States
(12) Patent Application Publication
(10) Pub. No.: US 2025/0340581 A1
(43) Pub. Date: Nov. 6, 2025

(54) PAEONIFLORIN MONOHYDRATE CRYSTAL FORM A, PREPARATION METHOD THEREFOR, AND USE THEREOF

(71) Applicant: SHANGHAI INSTITUTE OF MATERIA MEDICA.CHINESE ACADEMY OF SCIENCES, Shanghai (CN)

(72) Inventors: Yang YE, Shanghai (CN); Xuefeng MEI, Shanghai (CN); Sheng YAO, Shanghai (CN); Jianrong WANG, Shanghai (CN); Changqiang KE, Shanghai (CN); Shengqiang LI, Shanghai (CN); Chunping TANG, Shanghai (CN); Bintao HU, Shanghai (CN)

(21) Appl. No.: 19/267,714

(22) Filed: Jul. 14, 2025

Related U.S. Application Data
(63) Continuation of application No. PCT/CN2024/072267, filed on Jan. 15, 2024.

(30) Foreign Application Priority Data
Jan. 16, 2023 (CN) 202310057951.1

(51) Int. Cl. C07H 17/04; C07H 1/06

(52) U.S. Cl. CPC C07H 17/04; C07H 1/06

(57) ABSTRACT

The invention provides a paeoniflorin crystal form A and preparation method and application thereof. The crystal form A is characterized by means of XRD, IR, DSC, TGA and the like. The powder diffraction pattern of the crystal form A obtained by using an X-ray powder diffraction method of Cu Ka has diffraction peaks at the following angles of 2θ: 6.73°±0.2°, 7.94°±0.2°, 13.57°±0.2°, 15.53°±0.2°, 15.98°±0.2°, 17.16°±0.2°, 17.62°±0.2°, 20.45°±0.2°, 25.60°±0.2°. The paeoniflorin crystal form A of the present invention has low hygroscopicity and good physical stability, and is an ideal pharmaceutical crystal form.

### TECHNICAL FIELD

The invention belongs to the technical field of pharmaceutical solid chemistry, and particularly relates to a paeoniflorin monohydrate crystal form A and preparation method and application thereof.

### BACKGROUND

The chemical name of paeoniflorin is: β-D-Glucopyranoside, (1aR, 2S, 3aR, 5R, 5aR, 5bS)-5b-[(benzoyloxy) methyl] tetrahydro-5-hydroxy-2-methyl-2,5-methano-1H-3,4-dioxacyclobuta [cd] pentalen-1a(2H)-yl), molecular formula C₂₃C₂₈C₁₁, molecular weight 480.45, chemical structural formula:

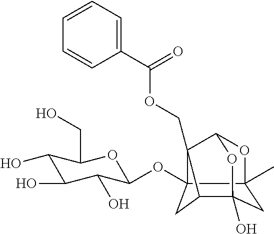 [SMILES: CC12CC3(O)OC(O1)C1(COC(=O)c4ccccc4)C3CC21OC1OC(CO)C(O)C(O)C1O]

Paeoniflorin is a pinane monoterpene glycoside compound, mainly found in the roots of Paeonia lactiflora, Paeonia suffruticosa, and other Paeonia lactiflora plants of the Ranunculaceae family. Paeoniflorin has various effects of tranquilizing, resisting inflammation, relieving pain, regulating immunity, improving cognitive ability, etc., and can be widely used in basic research of nervous system diseases and neurodegenerative diseases such as senile dementia, apoplexy, depression, epilepsy, etc. Researches in recent years show that paeoniflorin has a potential therapeutic effect on tumors and metastasis of colorectal cancer and angiogenesis-related diseases, and AbdEl-Aal and other researches find that paeoniflorin has an anti-angiogenesis effect and is expected to become a potential therapeutic drug for chronic hepatic fibrosis and angiogenesis-related diseases, so that advanced severe complications, tumors and metastasis are prevented (Exp Parasitol, 2019, 197, 85-92.). In addition, the paeoniflorin is reported to have better protective effect on carbon tetrachloride-induced acute liver injury and hepatic fibrosis (genomics and applied biology, 2018, 37(8): 3693-3698), and Ischemia-reperfusion-induced liver injury (Am J Transl Res, 2018, 10(3): 1012-1021.), also has obvious protective effect on SH-SYSY injury induced by rotenone (China J. Clin Pharmacology, 2018, 34(10): 1187-1190.); also has better effect on preventing and treating Diabetic Nephropathy (DN) and Diabetic Retinopathy (DR) (Biosci Trends, 2018, 12(2): 168-176), and the like.

Paeoniflorin is amorphous white powder with high hygroscopicity, and is easy to absorb moisture during storage, which affects the drug development of paeoniflorin. The research on the medical application of paeoniflorin is being effectively promoted, however, the polymorphism research thereof is relatively lagged, and the Cambridge crystal database does not record any relevant crystal data of paeoniflorin for many years, and the high water solubility and hygroscopicity of the paeoniflorin can be reasons for the difficulty in obtaining crystals.

Polymorphism refers to the phenomenon of a substance forming 2 or more solid existing forms due to differences in the conformation or arrangement of molecules in a crystal lattice. The definition of polymorphism can be divided into two ways, narrow and broad. Polymorphism in a narrow sense means that the chemical composition of a substance is the same, but there is a difference in the arrangement of molecules of the substance in a crystal. Polymorphism in a broad sense also includes amorphous forms and solvates (pseudopolymorphic forms). Polymorphism is very common for small molecule compounds. Polymorphism is also present in Active Pharmaceutical Ingredients (APIs) and is also common. Different crystal forms of a polymorphic drug often have different physicochemical properties, such as color, morphology, melting point, density, solubility, dissolution rate, compressibility, and the like. These differences in physicochemical properties will directly affect the bioavailability, stability, manufacturing process properties and even quality, safety and efficacy of the drug. If the attention on the problem of the crystal forms of a drug is neglected, the serious consequence is likely to be finally caused, and the development process of the drug is influenced.

Aiming at the problems of poor stability and high hygroscopicity of paeoniflorin in the storage process, the cultivation and discovery of a crystal form which can meet the relevant requirements of pharmaceutical preparation engineering and has bioavailability and physicochemical stability are urgently needed.

### SUMMARY

The inventor of the application continuously explores on the basis of comprehensively adopting a new crystallization nucleation mode and crystallization conditions, and discovers a new paeoniflorin crystal form named crystal form A. Researches show that the crystal form A has small hygroscopicity and good physical stability, has obvious advantages compared with the known amorphous form, and is in a more appropriate medicinal solid state.

The invention aims to provide a stable paeoniflorin monohydrate crystal form A, specifically, the paeoniflorin monohydrate crystal form A of the present invention is characterized in that, its X-ray powder diffraction pattern has diffraction peaks at the following angles of 2θ: 6.73°±0.2°, 7.94°±0.2°, 13.57°±0.2°, 15.53°±0.2°, 15.98°±0.2°, 17.16°±0.2°, 17.62°±0.2°, 20.45°±0.2°, 25.60°±0.2°.

In a preferred embodiment, the X-ray powder diffraction pattern of paeoniflorin monohydrate crystal form A further has diffraction peaks at the following angles of 2θ: 6.73°±0.2°, 7.94°±0.2°, 10.26°±0.2°, 10.67°±0.2°, 13.57°±0.2°, 15.53°±0.2°, 15.98°±0.2°, 17.16°±0.2°, 17.62°±0.2°, 18.72°±0.2°, 20.45°±0.2°, 25.60°±0.2°;

Preferably, the X-ray powder diffraction pattern has characteristic diffraction peaks at the following angles of 2θ: 6.73°±0.2°, 7.94°±0.2°, 10.26°±0.2°, 10.67°±0.2°, 13.57°±0.2°, 14.93°±0.2°, 15.53°±0.2°, 15.98°±0.2°, 17.16°±0.2°, 17.62°±0.2°, 18.72°±0.2°, 20.45°±0.2°, 21.47°±0.2°, 21.71°±0.2°, 22.28°±0.2°, 24.15°±0.2°, 25.37°±0.2°, 25.60°±0.2°, 27.39°±0.2°, 28.29°±0.2°, 31.20°±0.2°, 32.34°±0.2°, 32.86°±0.2°.

Preferably, the X-ray powder diffraction pattern has characteristic diffraction peaks at the following angles of 2θ: 6.73°, 7.94°, 10.26°, 10.67°, 13.57°, 14.93°, 15.53°, 15.98°, 17.16°, 17.62°, 18.72°, 20.45°, 21.47°, 21.71°, 22.28°, 24.15°, 25.37°, 25.60°, 27.39°, 28.29°, 31.20°, 32.34°, 32.86°.

In a preferred embodiment, form a is monoclinic, space group is P21, unit cell parameters are: a=11.0212(3) Å, b=8.4530(2) Å, c=12.9201(3) Å, α=90°, β=92.0860°, γ=90°, unit cell volume is 1202.87 Å³.

In other words, the present invention provides a paeoniflorin monohydrate crystal form A having an XRPD pattern substantially as shown in FIG. 1.

In a preferred embodiment, it is monoclinic, space group is P21, unit cell parameters are: α=11.0212(3) Å, b=8.4530(2) Å, c=12.9201(3) Å, α=90°, β=92.0860°, γ=90°, unit cell volume 1202.87 Å³.

In a preferred embodiment, the differential scanning calorimetry thermogram thereof has characteristic endothermic peaks at 125.8° C.±3.00° C. respectively, preferably the differential scanning calorimetry thermogram thereof is substantially as shown in FIG. 2, preferably the thermogravimetric analysis chart thereof is substantially as shown in FIG. 3, and preferably the infrared spectrum (IR) diagram thereof is substantially as shown in FIG. 4.

The invention provides a preparation method of the paeoniflorin monohydrate crystal form A, characterized in that the preparation method is one of the following methods: Method (1) Cooling method: dissolving paeoniflorin in a solvent at the temperature of 20-90° C., slowly cooling to 0-19° C. to obtain a suspension, and removing the solvent to obtain the paeoniflorin monohydrate crystal form A; or dissolving paeoniflorin in solvent at 20-90° C., slowly volatilizing at room temperature to remove solvent to obtain the paeoniflorin monohydrate crystal form A;
Method (2) Suspension method: adding excessive penoniflorin into solvent at 25-50° C., suspending for 8-96 h, centrifuging the obtained suspension to obtain lower layer solid as crystal form A, or centrifuging the suspension to obtain supernatant, volatilizing at 25-90° C. to obtain the penoniflorin monohydrate crystal form A,

Preferably, the solvent used in the methods (1) and (2) is one or more selected from dichloromethane, chloroform, diethyl ether, n-hexane, n-heptane, methyl isobutyl ketone, tetrahydrofuran. Further preferably, the solvent used in the methods (1) and (2) is dichloromethane, chloroform.

The present invention provides a pharmaceutical composition comprising a therapeutically effective amount of paeoniflorin monohydrate crystal form A according to any one of claims 1 to 5 and a pharmaceutically acceptable carrier. In particular to a pharmaceutical composition comprising a therapeutically effective amount of paeoniflorin monohydrate crystal form A and medicinal auxiliary materials. In general, a therapeutically effective amount of paeoniflorin monohydrate crystal form A is mixed or contacted with one or more pharmaceutical excipients to prepare a pharmaceutical composition or formulation, which is prepared in a manner well known in the pharmaceutical art.

The pharmaceutical composition can be prepared into a certain dosage form and is administrated by a proper route. For example, oral, parenteral (including subcutaneous, intramuscular, intravenous or intradermal), rectal, transdermal, nasal, vaginal, and the like. Formulations suitable for oral administration include tablets, capsules, granules, powders, pills, powders, lozenges, solutions, syrups or suspensions, as required, for the rapid, delayed or modified release of the active ingredient; formulations suitable for parenteral administration include aqueous or non-aqueous sterile injection solutions, emulsions or suspensions; formulations suitable for rectal administration include suppositories or enemas; formulations suitable for transdermal administration include ointments, creams, patches; formulations suitable for nasal administration include aerosols, sprays, nasal drops; formulations suitable for vaginal administration include suppositories, tampons, gels, pastes or sprays. Preferably, the crystal forms of the present invention, due to their unexpectedly low hygroscopicity and stability in water or aqueous ethanol, are particularly suitable in the preparation of tablets, suspensions, capsules, disintegrating tablets, immediate release, sustained release and controlled release tablets; further preferred are tablets, suspensions and capsules.

Pharmaceutically acceptable excipients in the above pharmaceutical compositions, in the case of solid oral dosage forms, include, but are not limited to: diluents, such as starch, pregelatinized starch, lactose, powdered cellulose, microcrystalline cellulose, calcium hydrogen phosphate, tricalcium phosphate, mannitol, sorbitol, sugar, and the like; binders, such as gum arabic, guar gum, gelatin, polyvinylpyrrolidone, hydroxypropyl cellulose, hydroxypropyl methylcellulose, polyethylene glycol, and the like; disintegrants, such as starch, sodium starch glycolate, pregelatinized starch, crospovidone, croscarmellose sodium, colloidal silicon dioxide and the like; lubricants, such as stearic acid, magnesium stearate, zinc stearate, sodium benzoate, sodium acetate, and the like; glidants, such as colloidal silicon dioxide and the like; complex-forming agents, such as various grades of cyclodextrins and resins; release rate controlling agents, such as hydroxypropyl cellulose, hydroxymethyl cellulose, hydroxypropylmethyl cellulose, ethyl cellulose, methyl cellulose, methyl methacrylate, wax and the like. Other pharmaceutically acceptable excipients that may be used include, but are not limited to, film forming agents, plasticizers, coloring agents, flavoring agents, viscosity modifying agents, preservatives, antioxidants, and the like. Optionally, the tablets are coated with a coating, for example by providing a shellac barrier coating, sugar coating or polymer coating, the polymers in the coating such as hydroxypropylmethyl cellulose, polyvinyl alcohol, ethyl cellulose, methacrylic polymers, hydroxypropyl cellulose or starch, may also include anti-sticking agents such as silica, talc, opacifying agents such as titanium dioxide, coloring agents such as iron oxide based coloring agents. In the case of liquid oral dosage forms, suitable excipients include water, oils, alcohols, glycols, flavoring agents, preservatives, stabilizers, coloring agents and the like; aqueous or non-aqueous sterile suspensions may contain suspending agents and thickening agents; suitable excipients for aqueous suspensions include synthetic or natural gums such as gum arabic, gum xanthium, alginate, dextran, sodium carboxymethylcellulose, methylcellulose, polyvinylpyrrolidone or gelatin. In the case of parenteral dosage forms, the excipients for aqueous or nonaqueous sterile injection solutions are generally sterile water, physiological saline, or aqueous dextrose, and may contain buffers, antioxidants, bacteriostats, and solutes which render the pharmaceutical composition isotonic with the blood. Each excipient must be acceptable, compatible with the other ingredients of the formulation and not deleterious to the patient. When used in the preparation of emergency drugs for cerebral apoplexy, powder injection is preferably considered.

The pharmaceutical composition may be prepared using methods well known to those skilled in the art. In preparing the pharmaceutical composition, the crystalline form A of the present invention is mixed with one or more pharmaceutically acceptable excipients, and optionally with one or more other active pharmaceutical ingredients. For example, tablets, capsules, granules can be prepared by processes of mixing, granulating, tableting, or encapsulating; powders are prepared by mixing pharmaceutically active ingredients and excipients ground to the appropriate size; solutions and syrups may be prepared by dissolving pharmaceutically active ingredients in suitably flavored water or aqueous solution; suspensions may be prepared by dispersing pharmaceutically active ingredients in pharmaceutically acceptable carriers. Particular mention is made of the wet granulation process of solid formulations, as for example of tablets, the preparation process being: mixing dry solids such as active ingredient, filler, binder, etc., wetting with a wetting agent such as water or alcohol, making the wetted solids into agglomerates or granules, continuing wet granulation until the desired uniform particle size is obtained, and then drying the granular product. Then mixing the obtained dry granules with disintegrating agent, lubricant, anti-sticking agent, etc., and tabletting in a tabletting machine; optionally, coating is carried out with a suitable coating powder.

The present invention provides use of the paeoniflorin monohydrate crystal form A in the preparation of a medicament for treating inflammation-related diseases or nervous system diseases including, but not limited to, senile dementia, parkinson's disease, epilepsy, huntington's disease, cerebral apoplexy, depression pain, acute myocardial ischemia, atherosclerosis, acute liver injury and hepatic fibrosis, diabetic nephropathy, and diabetic retinopathy, etc.

A large number of reports have shown that paeoniflorin has a definite curative effect on inflammation inhibition effect and can reduce the occurrence of inflammatory storm, such diseases include but are not limited to acute cerebral ischemia, inflammatory storm caused by new coronavirus and the like, and the damage of the inflammatory storm to the body of the user can be reduced. Therefore, the present invention provides use of the paeoniflorin monohydrate crystal form A in the preparation of a medicament for treating diseases related to inflammatory storm.

Compared with the prior art, the invention has the following excellent technical effects:

It should be noted that the inventors have obtained more than one paeoniflorin crystal form in the laboratory, the original experimental file is stored in the applicant's experimental file, and the other paeoniflorin crystal forms have a large difference from the crystal form A provided by the present invention in terms of excellent hygroscopicity and solubility, so specific parameters of the crystal forms are not recorded in the present specification. In all crystal forms, the crystal form A provided by the invention has good stability and low hygroscopicity, and can well avoid crystal transformation in the processes of medicament storage and development, thereby avoiding the change of bioavailability and medicament effect. Meanwhile, the crystal form A provided by the invention has higher solubility, meets the requirements of bioavailability and drug effect, can be suitably prepared into a water injection or powder injection preparation, and can be used for emergency treatment.

### BRIEF DESCRIPTION OF FIGURES

FIG. 1 is a graph of X-ray powder diffraction (XRPD) of paeoniflorin monohydrate crystal form A of example 1;

FIG. 2 is a Differential Scanning calorimetry (DSC) plot of paeoniflorin monohydrate crystal form A of example 1;

FIG. 3 is a graph of thermogravimetric analysis (TG) of paeoniflorin monohydrate crystal form A of example 1;

FIG. 4 is a graph of the infrared spectrum (IR) of paeoniflorin monohydrate crystal form A of example 1;

FIG. 5 is a crystal structure diagram of paeoniflorin monohydrate crystal form A;

FIG. 6 is a graph comparing X-ray powder diffraction (XRPD) of paeoniflorin monohydrate crystal form A with amorphous form;

FIG. 7 is a graph comparing hygroscopicity of the paeoniflorin monohydrate crystal form A of example 1 with amorphous form;

FIG. 8 is a graph of X-ray powder diffraction (XRPD) of paeoniflorin monohydrate crystal form B of comparative example 1.

FIG. 9 is a differential scanning calorimetry (DSC) plot of paeoniflorin monohydrate crystal form B of comparative example 1;

FIG. 10 is a graph of thermogravimetric analysis (TG) of paeoniflorin monohydrate crystal form B of comparative example 1;

FIG. 11 is a graph of the infrared spectrum (IR) of paeoniflorin monohydrate crystal form B of comparative example 1.

### DETAILED DESCRIPTION

The present invention is described in detail below by way of examples, but is not meant to be limited in any way. The compounds of the present invention may be prepared by a variety of synthetic methods well known to those skilled in the art, including the specific embodiments listed below, embodiments formed by combinations thereof with other chemical synthetic methods, and equivalents thereof known to those skilled in the art, with preferred embodiments including, but not limited to, examples of the present invention. It will be apparent to those skilled in the art that various changes and modifications can be made in the specific embodiments of the present invention without departing from the spirit and scope of the invention.

The term “effective therapeutically amount” or “Effective therapeutic dose” as used herein, refers to the amount of active compound or pharmaceutical agent that elicits the biological or medicinal response that is being sought by a researcher, veterinarian, medical doctor or other clinician in a tissue, system, animal, individual or human.

The term “treating” as used herein refers to one or more of the following: (1) prevention of disease; for example, preventing a disease, condition, or disorder in a subject who may be predisposed to the disease, condition, or disorder, but does not yet suffer from or display a pathology or symptomatology of the disease; (2) inhibition of disease; for example, inhibiting the disease, condition or disorder in an individual who is suffering from or displays a pathology or symptomatology of the disease, condition or disorder; and (3) amelioration of disease; for example, ameliorating the disease, condition, or disorder (i.e., reversing the pathology and/or symptomatology) in an individual suffering from or exhibiting the pathology and/or symptomatology of the disease, condition, or disorder, e.g., reducing the severity of the disease.

The term “polymorph” as used herein refers to different crystal forms of the same compound and includes, but is not limited to, other solid state molecular forms comprising hydrates and solvates of the same compound. The phenomenon that one drug molecule forms multiple crystal forms is called drug polymorphism, which is a ubiquitous phenomenon in solid drugs. Pharmaceutical compounds having such polymorphic forms are known to have an influence on pharmacological activity, solubility, bioavailability, stability and the like due to their physicochemical properties. Therefore, when a polymorph exists as a compound useful as a pharmaceutical, it is desired to produce a highly useful crystalline compound from the polymorph.

The term “X-ray powder diffraction pattern” as used herein refers to an experimentally observed diffraction pattern or a parameter derived therefrom. The X-ray powder diffraction pattern is characterized by the peak position and peak intensity.

The term “crystal” or “crystal form” as used herein refers to the form as characterized by the X-ray diffraction pattern characterization shown. It will be appreciated by those skilled in the art that the physicochemical properties discussed in the present invention can be characterized, with experimental error depending on the conditions of the instrument, sample preparation and purity of the sample. In particular, it is well known to those skilled in the art that the X-ray diffraction pattern will generally vary with the conditions of the instrument. It is particularly noted that the relative intensities of the X-ray diffraction pattern may also vary with the experimental conditions, so that the order of the peak intensities cannot be the only or decisive factor. In addition, experimental errors in peak angles are typically 5% or less, and these angles should also be taken into account, typically allowing errors of ±0.2°. In addition, due to the influence of experimental factors such as sample height, an overall shift in peak angle is caused, and a certain shift is usually allowed. Thus, it will be understood by those skilled in the art that the X-ray diffraction pattern of one of the crystal forms of the present invention need not be identical to the X-ray diffraction pattern of the examples referred to herein. Any crystal form having the same or similar pattern as the characteristic peaks in these patterns is within the scope of the present invention. One skilled in the art can compare the profiles listed in the present invention with a profile of an unknown crystal form to confirm whether the two sets of profiles reflect the same or different crystal forms.

In order to describe the present invention in more detail, the following preparation examples are given. Although the scope of the invention is not limited in this respect. Unless otherwise indicated, the methods and apparatus employed in the present invention are conventional in the art.

The experimental conditions involved in the experiments of the present invention are as follows:

The invention relates to a new paeoniflorin crystal form A, which is characterized by solid-state methods such as X-ray powder diffraction (XRPD), thermogravimetric analysis (TG), differential scanning calorimetry (DSC), infrared spectrum (IR), single-crystal X-ray diffraction (SCXRD) and the like.

XRPD analysis: the measurement was carried out at room temperature using a diffractometer of Bruker D8 advanced model, Bruker instruments, Germany, using Cu ka ray (λ=1.5418 Å), scanning at an angle of 20 from 3° to 40° and a scanning speed of 0.1°/s.

In the sample powder X-ray powder diffraction pattern, the diffraction pattern obtained from a particular crystal form is often characteristic. Because of differences in crystallization conditions, particle size, relative amounts of mixture, and other test conditions, diffraction patterns may produce preferential orientation effects, resulting in changes in the relative intensities of certain bands (especially at low angles) in the pattern. Therefore, the relative intensities of the diffraction peaks are not characteristic of the crystal concerned, and when judging whether the crystals are the same as the known crystal forms, the positions of the peaks should be noted rather than the relative intensities thereof. In addition, when determining whether the crystal forms are the same, attention should be paid to maintain the holistic concept because not one diffraction line represents one phase, but one specific set of “d-|/|1” data represents one phase. It should also be noted that in the identification of mixtures, the absence of a portion of the diffraction lines may be due to, for example, a reduction in the content, in which case it is not necessary to rely on all the bands observed in a high-purity sample, and even one band may be characteristic of a given crystal.

DSC analysis: the method adopts a DSC 8500 model differential scanning calorimeter of PerkinElmer company, US. for detection, the atmosphere is nitrogen, and the heating speed is 10° C./min.

TG analysis: the method adopts a thermogravimetric analyzer Netzsch TG 209F3 model of German Nachi company, temperature range: 30-400° C., scan rate: 10° C./min, purge gas: 25 mL/min.

IR analysis: the detection is carried out at room temperature by a Nicolet-Magna FT-IR 750 infrared spectrometer of Nicolet company, US., and the detection range is 4000-350 cm⁻¹ wave number.

SCXRD analysis: all the experimental data of single crystal X-ray diffraction (SCXRD) in this experiment were measured by Bruker D8 Venture model X-ray single crystal diffractometer, Bruker Instrument Company. The measurement conditions were as follows: graphite monochromator, Cu-kα ray (λ=1.54178 Å); temperature: room temperature (296K); voltage: 50 kV; current: 30 mA.

DVS analysis: all dynamic moisture sorption analysis (SCXRD) of this experiment were determined by DVS advantage model sorption instrument, British SMS company, relative humidity range: 0-95%, temperature: 25° C.

Specific examples of the preparation method and performance test of the crystal form A of the present invention are described below.

### Example 1

1 g of paeoniflorin (laboratory-made, batch number 98% PF_20180930, purity ≥98%) was uniformly mixed in 40 ml of ethyl acetate, and a magnetic stirrer was added for stirring at the rotating speed of 800 rpm and can be completely dissolved at the temperature of 50° C. Continued to stir and cool to 4° C. to get a suspension, filter, and dry under reduced pressure at room temperature. Form A was obtained as a white to off-white crystalline powder 0.91 g with a yield of 91%.

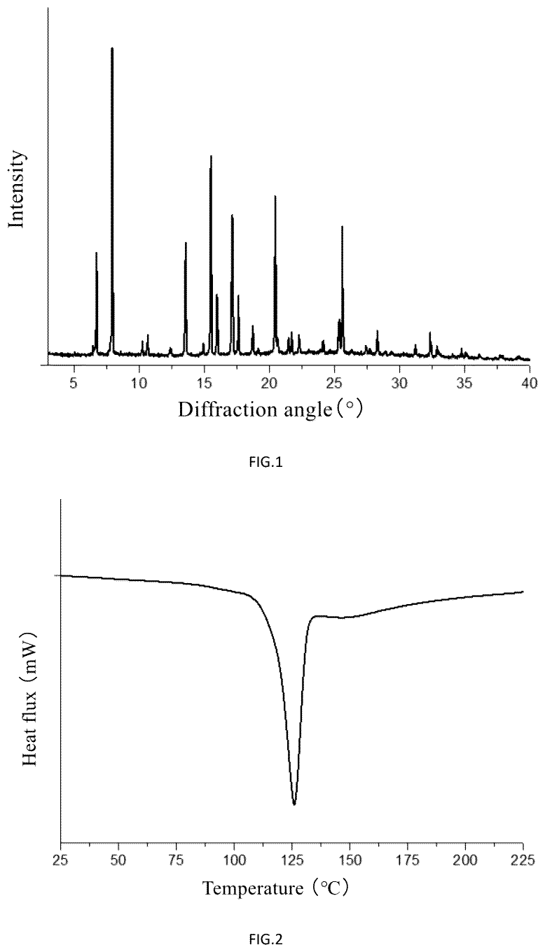

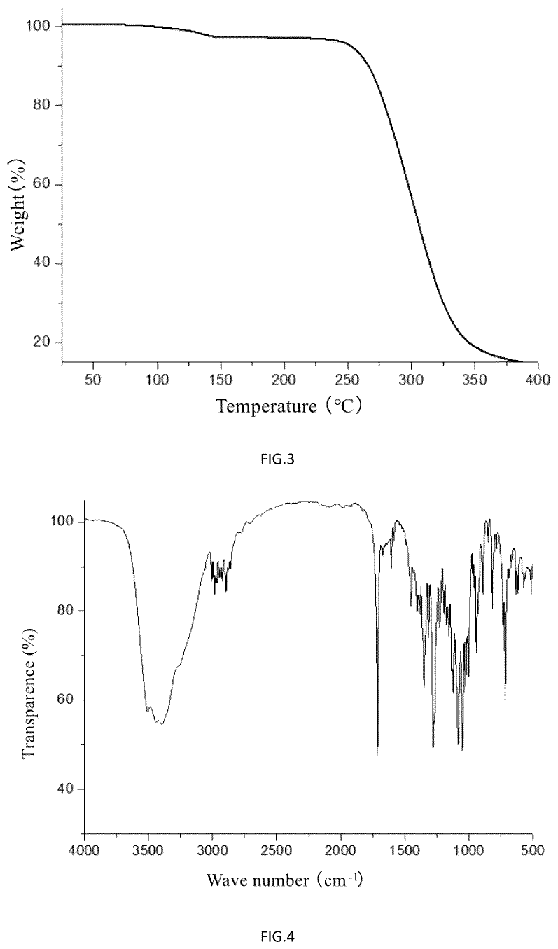

The X-ray powder diffraction (XRPD) pattern of paeoniflorin monohydrate crystal form A of example 1 is shown in FIG. 1; the thermogravimetric analysis (TG) diagram is shown in FIG. 2; the differential scanning calorimetry (DSC) profile is shown in FIG. 3; the Infrared (IR) spectrum is shown in FIG. 4. The water of crystallization comes from water molecules in air or solvent.

The single crystal X-ray diffractometer showed that paeoniflorin monohydrate crystal form A of example 1 is a monoclinic system with a space group of P21, unit cell parameters: α=11.0212(3) Å, b=8.4530(2) Å, c=12.9201(3) Å, α=90°, β=92.0860°, γ=90°, unit cell volume 1202.87 Å³.

Form A did not convert to amorphous or other forms and was very stable after 15 days acceleration in a stability box (40° C./75% RH).

TG and DSC showed that the phase transition and weight loss temperatures were both above 100° C., and the thermal stability was also excellent.

The crystal form A has small hygroscopicity, no wet agglomeration occurs, and the powder has good fluidity.

The crystal form A has good water solubility, the solubility in water is more than 20 mg/mL, and the crystal form A is easy to dissolve.

### Example 2

1 g of paeoniflorin was uniformly mixed in 10 ml of methyl ethyl ketone, a magnetic stirrer was added for stirring at the rotating speed of 800 rpm, suspended at room temperature for 12 h to obtain suspension, filtered, and dried at 20-30° C. under reduced pressure. Form A was obtained as a white to off-white crystalline powder 0.93 g with a yield of 93%.

The resulting product has an X-ray powder diffraction (XRPD) pattern similar to that of FIG. 1.

### Example 3

1 g of paeoniflorin was uniformly mixed in 10 mL of methyl ethyl ketone/n-hexane (v/v, 1:1), and a magnetic stirrer was added for stirring at the rotating speed of 800 rpm, suspended at room temperature for 12 h to obtain suspension, filtered, and dried at 20-30° C. under reduced pressure. Form A was obtained as a white to off-white crystalline powder 0.96 g with a yield of 96%.

The resulting product has an X-ray powder diffraction (XRPD) pattern similar to that of FIG. 1.

### Example 4

1 g of paeoniflorin was uniformly mixed in 10 ml of tetrahydrofuran/n-heptane (v/v, 1:1), and a magnetic stirrer was added for stirring at the rotating speed of 800 rpm, suspended at room temperature for 12 h to obtain suspension, filtered, and dried at 20-30° C. under reduced pressure. Form A was obtained as a white to off-white crystalline powder 0.95 g with a yield of 95%.

The resulting product has an X-ray powder diffraction (XRPD) pattern similar to that of FIG. 1.

### Example 5

500 mg paeoniflorin was added into 20 mL acetonitrile/n-hexane (v/v, 1:1), and a magnetic stirrer was added for stirring at the rotating speed of 600 rpm, and heated to 50° C. for complete dissolution. Slowly volatilized at 20-30° C., and dried under reduced pressure. Form A was obtained as a white to off-white crystalline powder 401 mg with a yield of 80%.

The resulting product has an X-ray powder diffraction (XRPD) pattern similar to that of FIG. 1.

### Example 6

500 mg of paeoniflorin was added into 20 mL acetonitrile/dichloromethane (v/v, 1:1), and a magnetic stirrer was added for stirring at the rotating speed of 600 rpm, heated to 50° C. for complete dissolution. Slowly volatilized at 20-30° C., and dried under reduced pressure. Form A was obtained as a white to off-white crystalline powder 405 mg with a yield of 81%.

The resulting product has an X-ray powder diffraction (XRPD) pattern similar to that of FIG. 1.

### Example 7

### Comparative Hygroscopicity Test

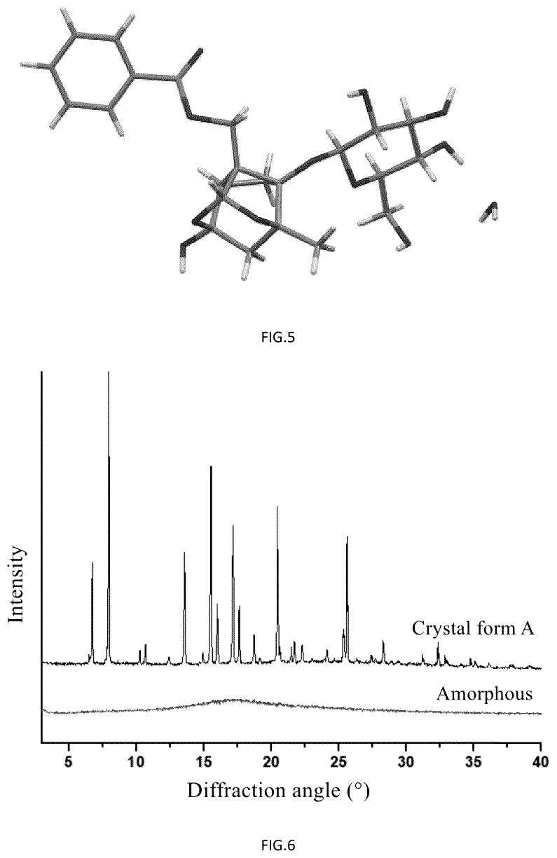

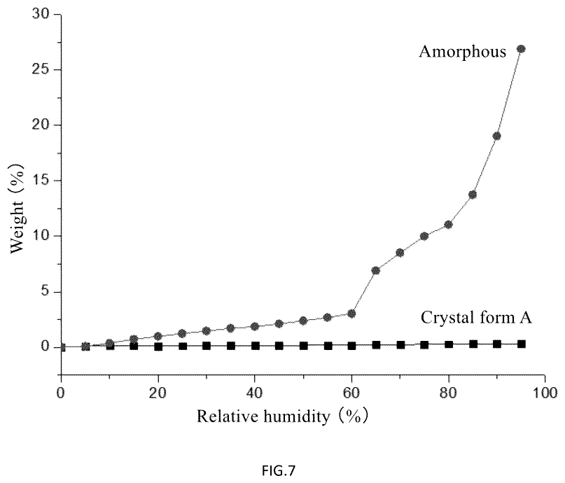

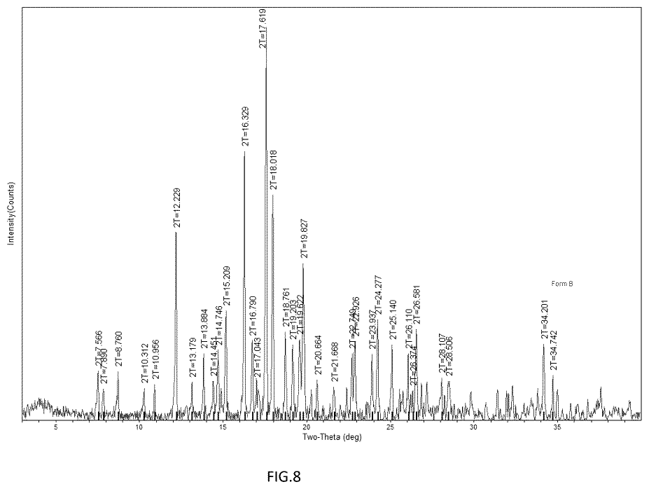

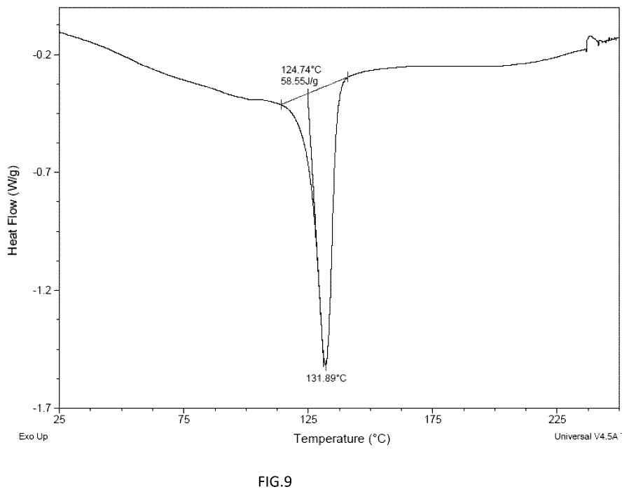

Commercially available paeoniflorin (Aladdin Company, batch number K2011169, purity ≥98%) was used as the amorphous powder, and its X-ray powder diffraction comparison diagram with the crystal form A of the present invention is shown in FIG. 6, and the hygroscopicity analysis comparison diagram of paeoniflorin and the crystal form A of the present invention is shown in FIG. 7. As can be seen from FIG. 7, the amorphous form adsorbs 11.1% moisture at 80% RH, indicating hygroscopicity; paeoniflorin monohydrate crystal form A of the present disclosure has an adsorbed moisture of only 0.3% at 80% RH.

### Comparative Example 1

During the process of cultivating crystal form A, another crystal form B of paeoniflorin was also found, and its preparation method is as follows:

In a 25 ml florence flask, 50 mg of paeoniflorin (laboratory-made, batch number 98% PF 20180930, purity >98%) was dissolved in 4 ml of methyl ethyl ketone, after the sample was completely dissolved, 4 ml of toluene was added and mixed evenly, the florence flask was left open at room temperature of 25° C. to slowly evaporate for 4 days until the solid fully precipitated. The suspension was filtered and dried under reduced pressure at room temperature. Crystal form B was obtained as 36 mg of white to off-white crystalline powder, with a yield of 72%.

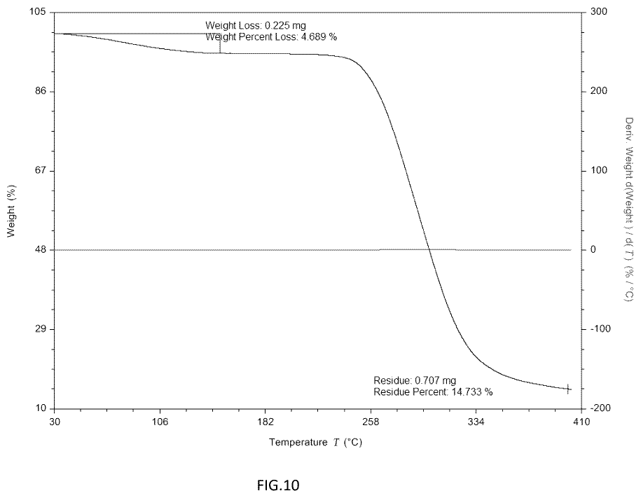

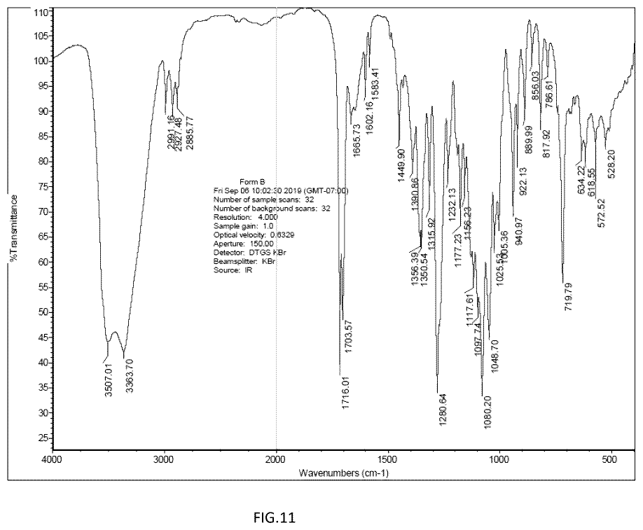

The X-ray powder diffraction (XRPD) pattern of crystal form B is shown in FIG. 8, and its basic physical properties are characterized as shown in FIGS. 9-11, it is a different crystal form from crystal form A.

### Comparative Example 2

### Another Preparation Method of Paeoniflorin Crystal Form B

In a 25 ml florence flask, 50 mg of paeoniflorin (laboratory-made, batch number 98% PF 20180930, purity >98%) was dissolved in 4 ml of ethyl ethyl ketone, after the sample was completely dissolved, 4 ml of methyl isobutyl ketone was added and mixed evenly, the florence flask was left open at 25° C. room temperature to slowly evaporate for 4 days until the solid fully precipitated. The suspension was filtered and dried under reduced pressure at room temperature. Crystal form B was obtained as 39 mg of white to off-white crystalline powder, with a yield of 78%. The X-ray powder diffraction (XRPD) pattern of crystal form B is similar to FIG. 8.

### Comparative Example 3

### Another Preparation Method of Paeoniflorin Crystal Form B

In a 25 ml florence flask, 50 mg of paeoniflorin (laboratory-made, batch number 98% PF 20180930, purity >98%) was dissolved into 4 ml of methyl ethyl ketone, after the sample was completely dissolved, 4 ml of isopropyl acetate was added and mixed evenly, the florence flask was left open at 50° C. water bath to evaporate for 4 h until the solid fully precipitated. The suspension was filtered and dried under reduced pressure at room temperature. Crystal form B was obtained as 42 mg of white to off-white crystalline powder, with a yield of 84%. The X-ray powder diffraction (XRPD) pattern of crystal form B is similar to FIG. 8.

### Comparative Example 4

### Hygroscopicity Test of Paeoniflorin Crystal Form B

The hygroscopicity of crystal form B was analyzed using DVS advantage adsorption instrument of SMS company in the UK, the amorphous form adsorbed 11.1% of water at 80% RH, showing hygroscopicity; Paeoniflorin crystal form B of the present invention adsorbed 2.22% of water at 80% RH, but its hygroscopicity was obviously different from that of crystal form A.

Conclusion: The hygroscopicity of the paeoniflorin crystal form A of the present invention is significantly reduced compared to the amorphous form, so that the storage and transportation costs of paeoniflorin are greatly reduced. Crystal form A does not form wet agglomeration, and the powder has good flowability, which provides convenience for the formulation engineering and is also beneficial to the quality control and metering control in the formulation engineering. Generally speaking, the easily soluble crystal form has a certain hygroscopicity, the crystal form A provided by the present invention still shows water solubility when the hygroscopicity is very low, making it possible to prepare it into a powder injection preparation for emergency treatment. The inventors also obtained other crystal forms of paeoniflorin in the laboratory, and has a large difference from the crystal form A provided by the present invention in terms of excellent hygroscopicity and solubility, so specific parameters of these crystal forms are not recorded in this specification.

Not all crystal forms of paeoniflorin have suitable hygroscopicity, Although the hygroscopicity of crystal form B is lower than that of the amorphous form, its hygroscopicity is still not suitable as a raw material for pharmaceutical preparations.

The crystal form A of the invention shows the advantages of various physicochemical properties and is an ideal medicinal crystal form.

What is claimed is:

1. A paeoniflorin monohydrate crystal form A, characterized in that its X-ray powder diffraction pattern has diffraction peaks at the following angles of 2θ: 6.73°±0.2°, 7.94°±0.2°, 13.57°±0.2°, 15.53°±0.2°, 15.98°±0.2°, 17.16°±0.2°, 17.62°±0.2°, 20.45°±0.2°, 25.60°±0.2°.

2. The paeoniflorin monohydrate crystal form A according to claim 1, characterized in that the X-ray powder diffraction pattern of the paeoniflorin monohydrate crystal form A further has diffraction peaks at the following angles of 2θ: 6.73°±0.2°, 7.94°±0.2°, 10.26°±0.2°, 10.67°±0.2°, 13.57°±0.2°, 15.53°±0.2°, 15.98°±0.2°, 17.16°±0.2°, 17.62°±0.2°, 18.72°±0.2°, 20.45°±0.2°, 25.60°±0.2°;
Preferably, the X-ray powder diffraction pattern has characteristic diffraction peaks at the following angles of 2θ: 6.73°±0.2°, 7.94°±0.2°, 10.26°±0.2°, 10.67°±0.2°, 13.57°±0.2°, 14.93°±0.2°, 15.53°±0.2°, 15.98°±0.2°, 17.16°±0.2°, 17.62°±0.2°, 18.72°±0.2°, 20.45°±0.2°, 21.47°±0.2°, 21.71°±0.2°, 22.28°±0.2°, 24.15°±0.2°, 25.37°±0.2°, 25.60°±0.2°, 27.39°±0.2°, 28.29°±0.2°, 31.20°±0.2°, 32.34°±0.2°, 32.86°±0.2°.

3. The crystal form A according to claim 1, which is a monoclinic system with a space group of P21, the unit cell parameters are: a=11.0212(3) Å, b=8.4530(2) Å, c=12.9201(3) Å, α=90°, β=92.0860°, γ=90°, the unit cell volume is 1202.87 Å³.

4. A paeoniflorin monohydrate crystal form A having an XRPD pattern substantially as shown in FIG. 1.

5. The crystal form A according to claim 1, which is a monoclinic system with a space group of P21, the unit cell parameters are: a=11.0212(3) Å, b=8.4530(2) Å, c=12.9201(3) Å, α=90°, β=92.0860°, γ=90°, the unit cell volume is 1202.87 Å³.

6. The crystal form A according to claim 1, wherein the differential scanning calorimetry analysis diagram has characteristic endothermic peaks at 125.8° C.±3.00° C. respectively, preferably, the differential scanning calorimetry analysis diagram is substantially as shown in FIG. 2, preferably, the thermogravimetric analysis diagram is substantially as shown in FIG. 3, preferably, the infrared spectrum (IR) diagram is substantially as shown in FIG. 4.

7. A preparation method of the paeoniflorin monohydrate crystal form A according to claim 1, characterized in that the preparation method is one of the following methods:
Method (1) Cooling method: dissolving paeoniflorin in a solvent at the temperature of 20-90° C., slowly cooling to 0-19° C. to obtain a suspension, and removing the solvent to obtain the paeoniflorin monohydrate crystal form A; or dissolving paeoniflorin in solvent at 20-90° C., slowly volatilizing at room temperature to remove solvent to obtain the paeoniflorin monohydrate crystal form A;
Method (2) Suspension method: adding excessive penoniflorin into solvent at 25-50° C., suspending for 8-96 h, centrifuging the obtained suspension to obtain lower layer solid as crystal form A, or centrifuging the suspension to obtain supernatant, volatilizing at 25-90° C. to obtain the penoniflorin monohydrate crystal form A,
Preferably, the solvent used in the methods (1) and (2) is one or more selected from dichloromethane, chloroform, diethyl ether, n-hexane, n-heptane, methyl isobutyl ketone, tetrahydrofuran.

8. A pharmaceutical composition comprising a therapeutically effective amount of the paeoniflorin monohydrate crystal form A according to claim 1 and a pharmaceutically acceptable carrier.

9. Use of the paeoniflorin monohydrate crystal form A according to claim 1 in the preparation of a medicament for treating inflammation-related diseases or nervous system diseases including, but not limited to, senile dementia, parkinson's disease, epilepsy, huntington's disease, cerebral apoplexy, depressive pain, acute myocardial ischemia, atherosclerosis, acute liver injury and hepatic fibrosis, diabetic nephropathy, and diabetic retinopathy, etc.

10. Use of the paeoniflorin monohydrate crystal form A according to claim 1 in the preparation of a medicament for diseases associated with inflammatory storm.
